(19) United States
(12) Patent Application Publication
(10) Pub. No.: US 2021/0162153 A1
(43) Pub. Date: Jun. 3, 2021

(54) Systems And Methods For Monitoring Tracheotomy Patients

(71) Applicant: The Regents of the University of California, Oakland, CA (US)

(72) Inventors: David E. Conrad, San Francisco, CA (US); Romain Christophe Roux, Berkeley, CA (US)

(21) Appl. No.: 16/770,392

(22) PCT Filed: Dec. 6, 2018
(86) PCT No.: PCT/US2018/064212

§ 371 Date: Jun. 5, 2020

Related U.S. Application Data
(60) Provisional application No. 62/595,177, filed on Dec. 6, 2017.

(51) Int. Cl. A61M 16/04; A61M 16/08; A61M 16/00

(52) U.S. Cl. CPC A61M 16/04; A61M 16/08; A61M 16/0003; A61M 2205/18; A61M 2230/432; A61M 2230/50; A61M 2016/0036

(57) ABSTRACT

In some embodiments, a tracheotomy tube monitoring device of a monitoring system includes a tube mounting portion configured to mount to a tracheotomy tube, sensing means for monitoring respiration of a patient in which the tracheotomy tube is inserted, and alert means for alerting relevant persons when data collected by the sensing means indicate that there is a problem.

### CROSS-REFERENCE TO RELATED APPLICATION

This application claims priority to co-pending U.S. Provisional Application Ser. No. 62/595,177, filed Dec. 6, 2017, which is hereby incorporated by reference herein in its entirety.

### BACKGROUND

Tracheotomy procedures, in which a passageway is formed through an incision in the neck to create an airway, are common. When a tracheotomy is performed, a tracheotomy tube is typically passed through the passageway to maintain its patency and provide a secure airway.

A common concern with tracheotomies and tracheotomy tubes is that the tube will become dislodged. Such dislodgement can range in severity from the tube shifting out of position and creating an air leak to the tube becoming completely removed from the passageway. Another concern is that the tracheotomy tube will become occluded, for example, with mucus generated by the patient. These situations pose a health risk to the patient and, potentially, a risk of death.

Given that tracheotomy tube dislodgement or occlusion may go unrecognized, it would be beneficial to have a means for monitoring the patient to ensure that the tracheotomy tube is in position and clear of obstruction.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a first perspective view of an embodiment of a tracheotomy tube monitoring device.

FIG. 2 is a second perspective view of the tracheotomy tube monitoring device of FIG. 1.

FIG. 3 is a first cross-sectional perspective view of the tracheotomy tube monitoring device of FIG. 1.

FIG. 4 is a second cross-sectional perspective view of the tracheotomy tube monitoring device of FIG. 1.

FIG. 5 is a perspective view of an embodiment of internal electrical components of the tracheotomy tube monitoring device of FIG. 1.

FIG. 6 is a side view of the tracheotomy tube monitoring device of FIG. 1 attached to a tracheotomy tube.

FIG. 7 is a cross-sectional perspective view of a further embodiment of a tracheotomy tube monitoring device.

FIG. 8 is a schematic view of a system for monitoring tracheotomy patients that includes a tracheotomy tube monitoring device and other devices configured to receive signals transmitted by the monitoring device.

### DETAILED DESCRIPTION

As can be appreciated from the discussion above, it would be desirable to have a system or method for monitoring a tracheotomy patient that can detect tracheotomy tube dislodgement or occlusion. Disclosed herein are examples of such systems and methods. In some embodiments, a monitoring system includes a tracheotomy tube monitoring device capable of monitoring patient respiration that attaches to the tracheotomy tube. In some embodiments, patient respiration is monitored using a conductive membrane of the device that is in contact with a sensing element of the device when the patient exhales and is pulled out of contact with the sensing element when the patient inhales. From this contact and non-contact, patient respiration can be monitored and analyzed. In other embodiments, patient is monitored using a pressure sensor in lieu of a conductive membrane. Such an embodiment is useful in cases in which the patient cannot tolerate the membrane, which functions in similar manner to a speaking valve. When no breathing or impaired breathing is detected, the device can generate an alert to warn appropriate persons. In some embodiments, the device emits an audible alarm and also wirelessly transmits an alert signal to an appropriate computing device, such as a smart phone and/or a computer.

In the following disclosure, various specific embodiments are described. It is to be understood that those embodiments are example implementations of the disclosed inventions and that alternative embodiments are possible. Such alternative embodiments include hybrid embodiments that include features of different disclosed embodiments. All such embodiments are intended to fall within the scope of this disclosure.

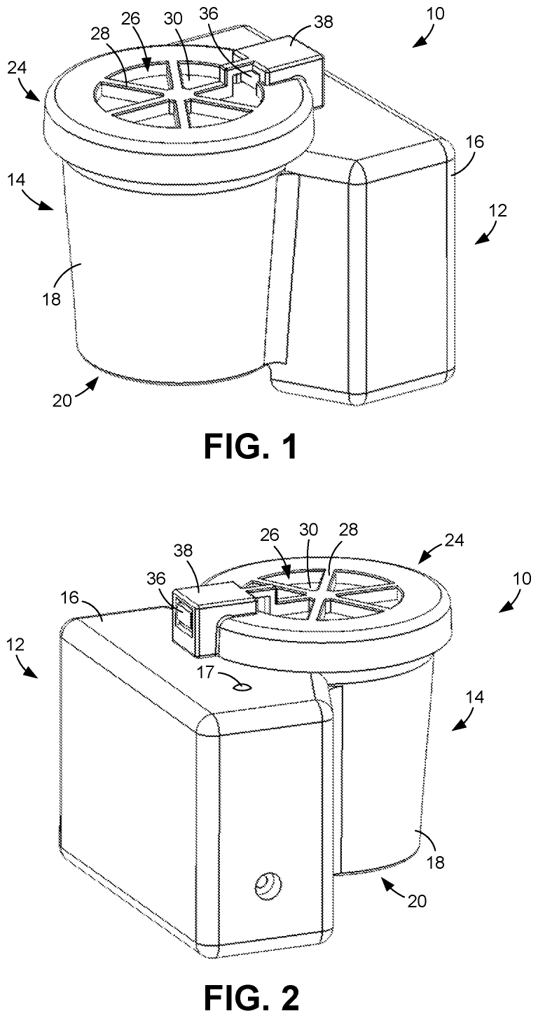

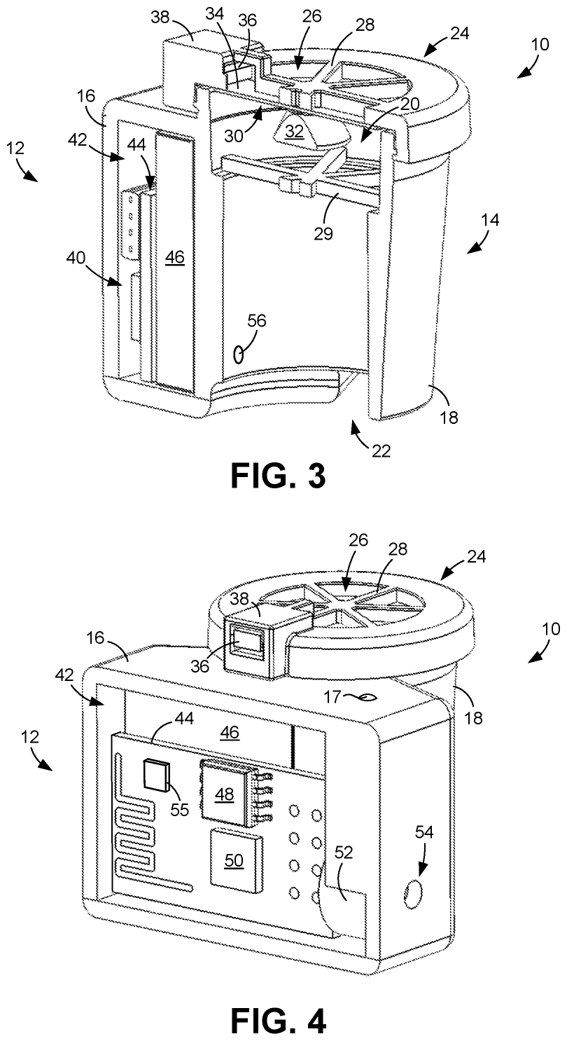

FIGS. 1 and 2 illustrate an embodiment of a tracheotomy tube monitoring device 10 that can comprise part of a system for monitoring tracheotomy patients. As shown in these figures, the illustrated monitoring device 10 includes a body portion 12 that contains internal electrical components of the device and a tube mounting portion 14 that is configured to mount the device to a tracheotomy tube. In this embodiment, the body portion 12 is formed as a generally rectangular housing 16. Provided on the housing 16 is an indicator 17, such as a light-emitting diode (LED) indicator light that illuminates when the device is activated. The tube mounting portion 14 is formed as a generally cylindrical airflow tube 18. As shown in FIG. 3, the airflow tube 18 is hollow and includes an inlet opening 20 and an outlet opening 22 through which air can flow. In some embodiments, the outlet opening 22 is sized and configured to receive the distal end of the tracheotomy tube with a press fit.

With reference to FIGS. 1-3, the tracheotomy tube monitoring device 10 further includes an end cap 24 that is mounted to the inlet end of the airflow tube 18. The end cap 24 includes a series of passages 26 through which air can flow. The passages 26 are defined by outer ribs 28 that extend radially outward from the center of the end cap 24 to its outer periphery. As is shown in FIG. 3, inner ribs 29 can be provided within the airflow tube 18 to provide structural integrity to the tube and to limit insertion of the tracheotomy tube.

With further reference to FIG. 3, positioned between the end cap 24 and the airflow tube 18 on the inner side of the outer ribs 28 is a flexible membrane 30, which can, for example, be made of a flexible elastomeric material, such as a polymer elastomer, silicone, or rubber. The center of the membrane 30 is secured to the end cap 24 with a generally conical retainer element 32 that extends from the center of the end cap. The edges of the membrane 30, however, are not secured to the end cap 24 and, therefore, can deform (i.e., bend inward) to enable air to pass through the end cap 24, past the membrane, and into the airflow tube 18 during patient inhalation.

At least a top side 34 of the membrane 30 is electrically conductive so that contact between the membrane and a sensing element 36 can be detected. This electrical conductivity can, for example, be provided by a thin conductive substrate that is applied to the membrane 30 or a conductive material that is deposited on the surface of the membrane. In some embodiments, the conductive substrate/material is a metal material, such as gold. The sensing element 36 can comprise a small circuit board having electrical contacts that are placed in contact with the top side 34 of the membrane 30 when the patient is not inhaling, thereby closing an electrical circuit. In the illustrated embodiment, the sensing element 36 is contained within a small housing 38 that forms part of the end cap 24.

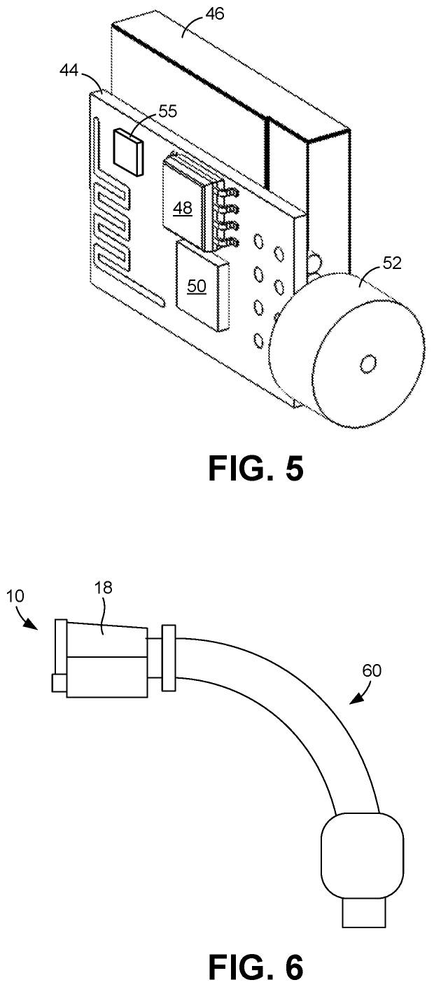

With reference next to FIGS. 3-5, the sensing element 36 is in electrical communication with internal electrical components 40 of the tracheotomy tube monitoring device 10 that are contained within an interior space 42 of the body portion 12. In the illustrated embodiment, these electrical components 40 include a circuit board 44 and a battery 46, which may be rechargeable. Mounted to the circuit board 44 is a microcontroller 48 configured to control the overall operation of the device 10, a wireless transceiver chip 50 configured to wirelessly transmit signals to other devices (e.g., via Bluetooth or WiFi), and a speaker 52 configured to generate an audible alarm, which can be emitted through an opening 54 provided in the body portion 12.

Also mounted to the circuit board 44 is an accelerometer 55 that is configured to sense vibrations transmitted by the tracheotomy tube to the monitoring device 10. Such vibrations can include those associated with patient breathing as well as those associated with the presence of an obstruction within the tracheotomy tube, such as a mucus plug. In some embodiments, the accelerometer 55 can also sense vibrations associated with other phenomena. For example, the accelerometer 55 may be capable of sensing beating of the patient's heart so that the monitoring device 10 can monitor patient heart rate. As another example, the accelerometer 55 may be capable of sensing patient movements, such as sitting up, walking, falling, and the like. It is noted that a microphone could be used in lieu of or conjunction with the accelerometer 55 to sense vibrations.

With reference back to FIG. 3, the monitoring device 10 can further include a connection sensor 56 that is configured to sense a physical parameter, such as pressure, that is indicative of a positive connection to a tracheotomy tube. If the connection sensor 56 does not sense such a connection, an alert can be generated.

As noted above, the tracheotomy tube monitoring device 10 is configured to be removably attached to a tracheotomy tube. In particular, as depicted in FIG. 6, the distal end of the tracheotomy tube 60 can be inserted into the airflow tube 18 of the monitoring device 10 until a snug (interference) fit is achieved and/or the tracheotomy tube abuts the inner ribs 29. Once the monitoring device 10 is so attached and then activated (e.g., using a switch or button provided on the device (not shown)), the device can monitor patient respiration with for purpose of detecting tracheotomy tube dislodgement and/or occlusion. The monitoring device 10 does this by detecting the cyclic contact and non-contact between the conductive membrane 30 and the sensing element 36.

When the patient inhales, air is drawn through the tracheotomy tube and creates a vacuum within the airflow tube 18 that causes the membrane 30 to separate from the sensing element 36 along its outer edges and enables air to flow around the membrane, through the airflow tube, and to the patient. When this separation occurs, the electrical connection between the sensing element 36 (which is positioned near the outer edge of the membrane 30) and the membrane is lost. When the patient exhales, positive pressure is applied to the membrane 30 within the airflow tube 18 to return the membrane to its original orientation in which it makes positive contact with the sensing element 36. In addition, the membrane 30 is pressed into contact with the ribs 28 at which point no air flows through the device 10. Accordingly, the end cap 24 and membrane 30 together function as a one-way valve that enables air to flow through the device 10 only during inhalation.

It is noted that the above form of operation also enables the device 10 to function as a speaking valve. In particular, when the patient exhales to speak, the membrane 30 is urged against the outer ribs 28 of the end cap 24 so as to close the passages 26 so that exhaled air will flow through the vocal cords instead of the device 10. In cases in which this functionality is not desired, holes (not shown) can be provided in the airflow tube 18 to enable exhaled air to escape from the device 10. Even in such a case, the membrane 30 is urged against the outer ribs 28 as there is still adequate air pressure within the airflow tube 18 to achieve this. Moreover, in some embodiments, the natural position for the membrane 30 is one in which it is in positive contact with the outer ribs 28 as the membrane naturally seats against the ribs. In that case, positive pressure is not necessary to create contact between the membrane 30 and the sensing element 36.

The microcontroller 48 monitors the signals received from the sensing element 36 (i.e., an open circuit condition during inhalation and a closed-circuit condition during exhalation) and uses these signals to evaluate the respiration of the patient. More particularly, the microcontroller 48 uses software or firmware (i.e., computer-readable, executable instructions, which may embody one or more algorithms) to analyze the rhythms of the patient's breathing. As long as the monitored signals are indicative of normal patient breathing, it is presumed that the tracheotomy tube is in proper position and is not occluded. If the signals are not indicative of normal patient breathing, however, an alert is generated as this condition may be indicative of improper tracheotomy tube placement and/or occlusion. As mentioned above, the alert can be an alarm that is emitted by the speaker 52 and/or an alert signal that is wirelessly transmitted to another device. In addition, the software/firmware can receive signals from the accelerometer 55 and the connection sensor 56 and issue alerts as necessary based upon the signals that are received (e.g., clogging of the tracheotomy tube, improper connection with the tracheotomy tube, etc.).

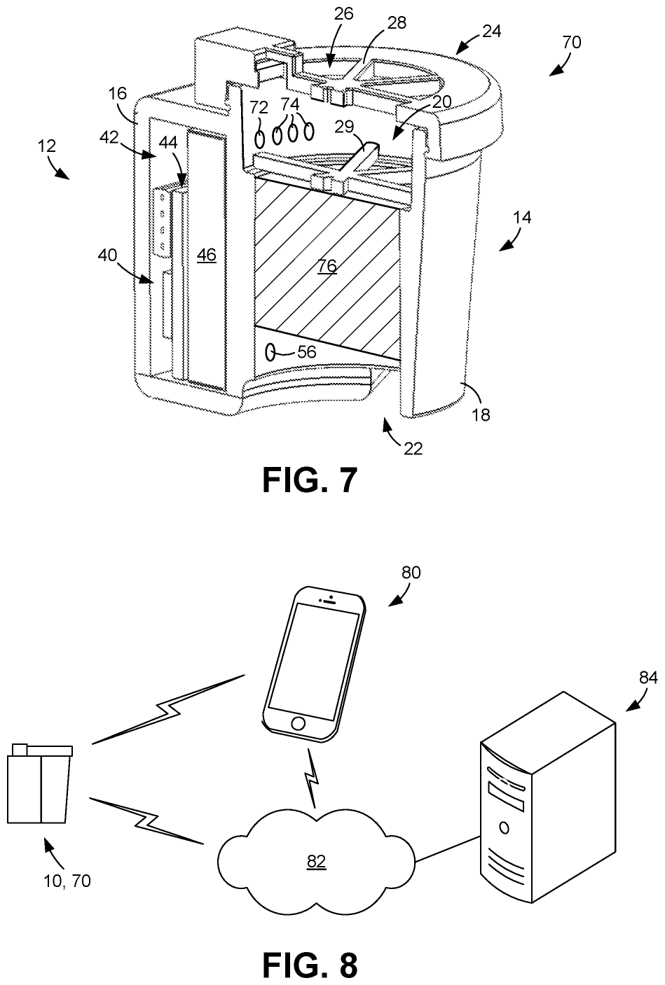

FIG. 7 illustrates a further embodiment of a tracheotomy tube monitoring device 70. The monitoring device 70 is similar in many ways to the monitoring device 10 shown in FIGS. 1-5. In some embodiments, the monitoring device 70 comprises nearly all of the same components of the monitoring device 10. Accordingly, the monitoring device 70 comprises a body portion 12 and a tube mounting portion 14 that includes an airflow tube 18. The monitoring device 70 further includes an end cap 24 having series of passages 26 through which air can flow. In this embodiment, however, the monitoring device 70 comprises no membrane configured to close when the patient exhales. Instead of a membrane and a sensing element that detects contact with the membrane, the monitoring device 70 comprises a pressure sensor 72 that is positioned within the airflow tube 18 (although a particular position for the pressure sensor 72 is shown in FIG. 7, it is noted that the pressure sensor, and other sensors described below, can be located in other positions within the airflow tube). The pressure sensor 72 can detect changes in pressure within the airflow tube 18 that are indicative of inhalation and exhalation. Accordingly, the pressure sensor 72 can monitor patient respiration as with the embodiment of FIGS. 1-5.

In addition to the pressure sensor 72, further sensors 74 can be provided within the airflow tube 18. Such can sensors include a temperature sensor configured to measure the temperature of the exhaled air, a humidity sensor configured to measure the humidity of the exhaled air, and a carbon dioxide sensor configured to measure the concentration of carbon dioxide in the exhaled air.

Another addition to the monitoring device 70 relative to the monitoring device 10 is the presence of a heat and moisture exchanger (HME) 76 within the airflow tube 18. As is known in the art, an HME is a component that traps moisture in the patient's exhaled air and humidifies the air that is inhaled by the patient. By providing moisture to the air that the patient inhales, the HME 76 reduces mucus formation and, therefore, reduces the likelihood for tracheotomy tube occlusion.

As identified above, the tracheotomy tube monitoring device can wirelessly transmit signals to other devices for purposes of issuing alerts to relevant parties. The monitoring device can be configured to transmit those signals to a variety of different devices. For example, as shown in FIG. 8, a monitoring device 10, 70 can be configured to transmit signals to a smart phone 80 or a computer network 82 to which one or more computers 82 are connected. In addition to receiving alerts, the other devices can receive the data collected by the monitoring device 10, 70 and perform analysis on that data. Such analysis can comprise analysis as to patient respiration as well as other phenomena relevant to the patient's health. For instance, data collected by the accelerometer 55 can be used to determine a variety of information about the patient's health as well as activity. In some embodiments, the various collected data can be used to generate an overall health score for the patient that provides a general indication as to how the patient is doing. In other embodiments, the monitoring system can include a dedicated receiver unit that can be placed in a location in which a person responsible for the patient, such as hospital staff or another caregiver, can hear and/or see an alert generated by the unit.

As noted above, various modifications can be made to the disclosed tracheotomy tube monitoring devices in accordance with the present disclosure. For example, the monitoring device can be modified for use with a respirator. Specifically, the monitoring device can be modified such that it can be inserted inline along a respirator tube that delivers air to a patient.

1. A tracheotomy tube monitoring device comprising:
a tube mounting portion configured to mount to a tracheotomy tube;
sensing means for monitoring respiration of a patient in which the tracheotomy tube is inserted; and
alert means for alerting relevant persons when data collected by the sensing means indicate that there is a problem.

2. The monitoring device of claim 1, wherein the tube mounting portion comprises an airflow tube configured to receive an end of the tracheotomy tube.

3. The monitoring device of claim 2, wherein the airflow tube comprises an inlet end and an outlet end.

4. The monitoring device of claim 3, further comprising an end cap mounted to the inlet end of the airflow tube.

5. The monitoring device of claim 1, wherein the sensing means comprise a conductive membrane associated with the tube mounting portion and a sensing element positioned in proximity to the conductive membrane, wherein the conductive membrane is urged into contact with the sensing element when a patient exhales through the tracheotomy tube and is drawn out of contact with the sensing element when the patient inhales through the tracheotomy tube.

6. The monitoring device of claim 5, wherein the conductive membrane is made of a flexible, elastic material and includes conductive material provided on one side of the membrane.

7. The monitoring device of claim 6, wherein the conductive material is comprised by a thin conductive substrate that is applied to the membrane.

8. The monitoring device of claim 6, wherein the conductive materials is deposited on a surface of the membrane.

9. The monitoring device of claim 1, wherein the sensing means comprise a pressure sensor that is provided within the airflow tube.

10. The monitoring device of claim 1, further comprising a microcontroller configured to receive data sensed by the sensing means and to generate alerts.

11. The monitoring device of claim 10, further comprising a speaker controlled by the microcontroller configured to emit an audible alarm.

12. The monitoring device of claim 10, further comprising a wireless transceiver controlled by the microcontroller configured to wirelessly transmit an alert signal to another device.

13. The monitoring device of claim 1, further comprising an accelerometer configured to sense vibrations transmitted through the tracheotomy tube to the monitoring device.

14. The monitoring device of claim 1, further comprising a connection sensor configured to sense positive connection between the monitoring device and the tracheotomy tube.

15. The monitoring device of claim 1, further comprising a temperature sensor and a humidity sensor proved within the airflow tube configured to sense a temperature and a humidity, respectively, of air exhaled by the patient.

16. The monitoring device of claim 1, further comprising a carbon dioxide sensor configured to sense a concentration of carbon dioxide of air exhaled by the patient.

17. The monitoring device of claim 1, further comprising a heat and moisture exchanger provided within the airflow tube configured to trap moisture in air exhaled by the patient and humidify air to be inhaled by the patient.

18. A system for monitoring a tracheotomy patient, the system comprising:
a tracheotomy tube monitoring device including a tube mounting portion configured to mount to a tracheotomy tube, sensing means for monitoring respiration of a patient in which the tracheotomy tube is inserted, and a wireless transceiver configured to wirelessly transmit an alert signal when data collected by the sensing means indicates that there is a problem; and
a computing device configured to receive the alert wirelessly transmitted by the wireless transceiver.

19. A method for monitoring a tracheotomy patient, the method comprising:
connecting a tracheotomy tube monitoring device to a tracheotomy tube inserted into a patient;
monitoring respiration of the patient with the monitoring device; and
the monitoring device generating an alert when data collected by the monitoring device indicates that there is a problem.

20. The method of claim 19, wherein generating an alert comprises emitting an audible alarm from the monitoring device, transmitting an alert signal to another device, or both.
